(19) United States
(12) Patent Application Publication
(10) Pub. No.: US 2005/0272802 A1
(43) Pub. Date: Dec. 8, 2005

(54) Process for preparing form I of tegaserod maleate

(76) Inventors: Sundaram Venkataraman, Hyderabad (IN); Srinivasulu Gudipati, Hyderabad (IN); Brahmeshwararao Mandava Venkata Naga, Hyderabad (IN); Goverdhan Reddy Banda, Hyderabad (IN); Radhakrishna Singamsetty, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/111,373

(22) Filed: Apr. 21, 2005

Related U.S. Application Data
(60) Provisional application No. 60/601,689, filed on Aug. 13, 2004.

(30) Foreign Application Priority Data
Apr. 22, 2004 (IN) 369/CHE/2004

(51) Int. Cl. A61K 31/405; C07D 209/14

(52) U.S. Cl. 514/419; 548/505

(57) ABSTRACT

A process for preparing Form I of tegaserod maleate comprises the steps of: (i) dissolving tegaserod in a solvent; (ii) optionally, subjecting the solution to activated carbon treatment; (iii) adding maleic acid solution to the tegaserod solution; and (iv) isolating crystalline Form I of tegaserod maleate.

### CROSS-REFERENCE TO RELATED APPLICATIONS

This application claims priority from copending U.S. Provisional Application 60/601,689 filed Aug. 13, 2004, and from India Patent Application No. 369/CHE/2004 filed Apr. 22, 2004. The entire content of each of the prior applications is hereby incorporated by this reference.

### INTRODUCTION TO THE INVENTION

The present invention relates to a simple and improved process for the preparation of crystalline form I of tegaserod maleate.

Tegaserod maleate, chemically known as 2-[(5-Methoxy-1H-indol-3-yl) -methylene] N-pentylhydrazine carboximidamide maleate, is represented as Formula (I), 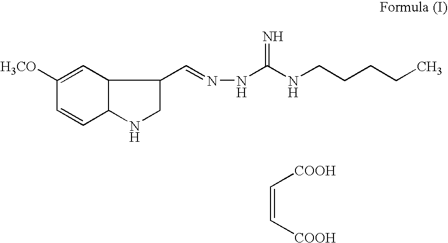
and is useful for treating gastrointestinal disorders, such as in the treatment of Irritable bowel syndrome (IBS) disease.

U.S. Pat. No. 5,510,353 discloses generically tegaserod and its maleate salt, pharmaceutical compositions, the preparation thereof, and their use in the treatment of gastrointestinal motility disorders such as irritable bowel syndrome.

CN 1425651 discloses a kind of tegaserod maleate containing water crystal with sound stability. The CN 1425651 describes two kinds of crystallizing forms of tegaserod maleate, anhydrous crystallization abbreviated as W-shaped crystallization and mono-crystallization abbreviated as S-shaped crystallization. CN 1425651 further describes the process for the preparation of tegaserod maleate in S-shaped crystallization, which involves the crystallization in organic solvents which can dissolve in water, such as 50%-95% ethanol, methanol, isopropyl alcohol, acetone, methyl cyanide, and dimethylformamide. The process for the preparation of tegaserod maleate in W-shaped crystallization involves heating the tegaserod maleate and methanol.

WO 2004/085393 describes the crystalline Form I, II, IlI, and IV of tegaserod maleate and a process for the preparation thereof. WO 2004/085393 describes the Form I of tegaserod maleate characterized by an X-ray powder diffraction pattern having peaks at 5.3, 5.9, 6.4, 10.7, 16.1 and 26.8 two theta degrees depicted in FIG. 1 of WO 2004/085393. The publication also describes the process for the preparation of Form I of tageserod maleate by adding maleic acid to a solution of tageserod base in acetone, followed by isolation or by refluxing form II of tageserod maleate in acetone.

The inventors of the present invention have been investigating the use of acetone as solvent at excess temperature and prolonged time in the preparation of Form I of tegaserod maleate, which results in the mixture of Form I and Form S during addition of maleic acid to solutions of tegaserod base in acetone.

Accordingly, there is a need for the preparation of Form I of tageserod maleate in a consistent manner.

### SUMMARY OF THE INVENTION

In accordance with one aspect, the present invention provides a process for the preparation of Form I of tegaserod maleate, in a consistent manner. An aspect of the process for the preparation of Form I of tegaserod maleate comprises the steps of:
i. dissolving tegaserod in a solvent;
ii. optionally, subjecting the solution to activated carbon treatment;
iii. adding maleic acid solution to the tegaserod solution; and
iv. isolating crystalline Form I of tegaserod maleate.

The process of the present invention is environmentally friendly and can be applied in large scale, e.g. on the kilogram level, for commercial manufacturing of Form I of tegaserod maleate.

### BRIEF DESCRIPTION OF THE DRAWINGS

FIG. 1 is an X-Ray powder diffractogram of tegaserod maleate Form I, as measured on Bruker Axe, DS Advance Powder X-ray Diffractometer with a Cu K alpha-1 radiation source.

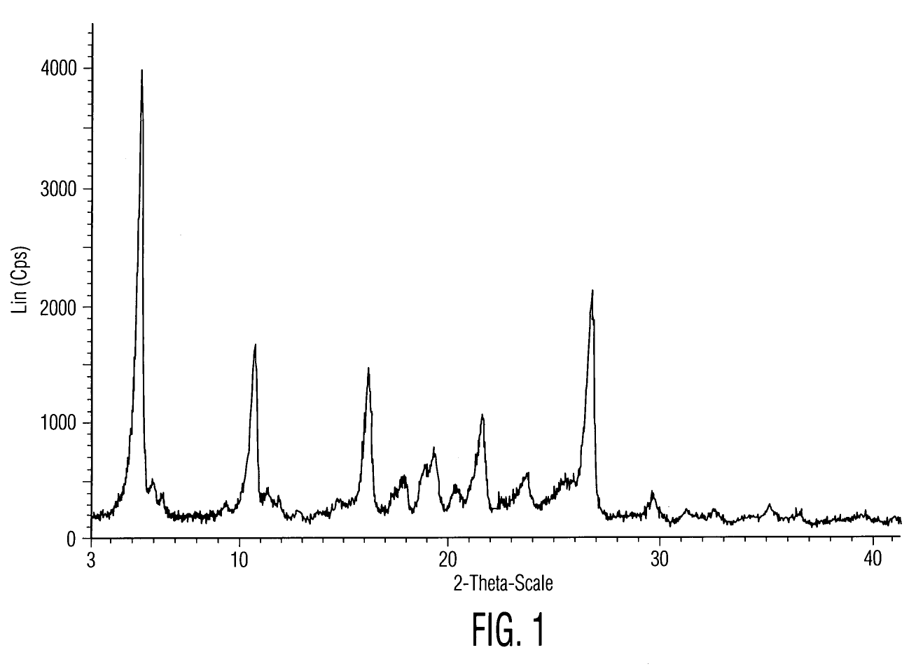

FIG. 2 is an infrared absorption spectrum of tegaserod maleate Form I.

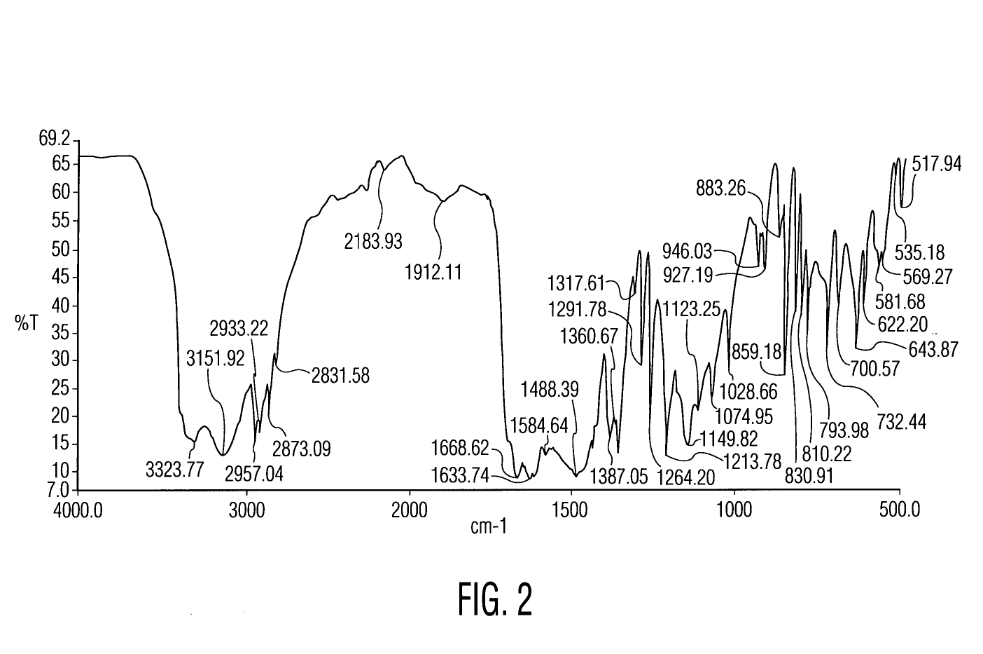

### DETAILED DESCRIPTION

It has been surprisingly found that the use of ethyl methyl ketone as solvent at excess temperature, variation in concentration, and prolonged time in the preparation of Form I of tageserod maleate does not result into the mixture of Forms I and S during addition of maleic acid to solutions of tegaserod base in ethyl methyl ketone.

The present invention provides a process for the preparation of Form I of tegaserod maleate, which does not contain the mixture of Forms I and S of tegesarod maleate.

Accordingly, the present invention provides the process for the preparation of Form I of tegaserod maleate, comprising the steps of;
i. dissolving tegaserod in a solvent;
ii. optionally, subjecting the solution to activated carbon treatment;
iii. adding maleic acid solution to the tegaserod solution; and
iv. isolating crystalline Form I of tegaserod maleate.

The present invention provides a process wherein tegaserod base is dissolved in a solvent. Useful solvents include ketones having the formula R₁C(O)R2 where R₁ is a straight or branched alkyl group having 1 to 4 carbon atoms, and R₂ is a straight or branched alkyl group having 2 to 4 carbon atoms. Preferably, the solvent comprises methyl isobutyl ketone, methyl isopropyl ketone, ethyl methyl ketone or mixtures thereof. A preferred solvent is ethyl methyl ketone. The dissolution is carried out at any temperatures, preferably at ambient temperature.

The optional carbon treatment is carried out at any temperature, preferably ambient temperature, to facilitate adsorption of impurities. The impurities are removed from the solution, and are filtered out along with carbon. The carbon will usually be washed with a solvent, preferably ethyl methyl ketone, and the solvent added to the solution.

Maleic acid is dissolved in a solvent to form the maleic acid solution. The solvent can be any of the ketones described above, preferably methyl isobutyl ketone, methyl isopropyl ketone, ethyl methyl ketone or mixtures thereof. Preferably the maleic acid solution is formed at temperatures below 50° C., preferably about 25 to 35° C., more preferably 27 to 29° C.

Maleic acid solution is added to the solution containing tegaserod at temperatures below 50° C., preferably about 20 to 45° C., more preferably about 25-35° C. The addition of maleic acid solution to the solution containing tegaserod is preferably done over about 5 minutes to about 90 minutes; preferably the addition step is conducted over at least about 20 minutes.

Preferably maleic acid solution is added to the solution containing tegaserod. The molar ratio of tegaserod to maleic acid is preferably about 1:1 to about 1:1.5, more preferably about 1:1.1.

The resultant suspension is cooled to about room temperature under stirring to form crystals of Form I of tegaserod maletae. The crystals of Form I of tegaserod maleate are filtered and washed with solvent. Preferably, crystals of Form I of tegaserod maleate are then dried. Any drying technique known to a person skilled in the art may be used. Preferably, drying under vacuum at about 25-45° C. for about 13 to 15 hours or overnight, followed by further drying under vacuum at 55 to 75° C. for about 3 to 4 hours, performs this drying step.

The process of the present invention is environmentally friendly and can be conducted in a large scale, such as to produce kilogram quantities, for commercial manufacturing of Form I of tegaserod maleate. The process for the preparation of Form I of tegaserod maleate of the present invention is simple, eco-friendly, industrially feasible, and economically inexpensive. Form I of Tegaserod maleate is well suited for pharmaceutical formulations and can be used in the treatment of gastrointestinal motility disorders such as irritable bowel syndrome.

The following examples are illustrative of the invention but are not intended to limit the scope of the claims in any way.

### EXAMPLE I

150 milliliters of ethyl methyl ketone and 5.0 grams tegaserod were taken into a round-bottom flask at a temperature of about 28° C. followed by stirring. 0.5 grams of carbon are added to the above reaction mixture with continuous stirring for about 5 to 15 minutes. The reaction mass that was obtained was then filtered and washed with 10 milliliters of ethyl methyl ketone. The filtrate was taken into a round-bottom flask, which was then subjected to heating at to temperature of about 38° C. with simultaneous stirring. 2.1 grams of maleic acid were dissolved in 25 milliliters of ethyl methyl ketone and the resulting solution was added to the above reaction mass at a temperature of about 44° C. over a period of about 30 minutes. The round-bottom flask was placed into a tub followed by stirring at a temperature of about 31° C., which is then filtered and washed with 10 milliliters of ethyl methyl ketone. The solid then obtained was suck dried at a temperature of about 30° C., transferred into a petri dish and subjected to air drying for about 14 hours to afford the compound of interest that is Form I of tegaserod maleate having a purity of 99.8%.

### EXAMPLE II

5.0 grams tegaserod and 125 milliliters of ethyl methyl ketone were taken into a round-bottom flask at a temperature of about 28° C. followed by stirring. 0.5 gram of carbon was added to the above reaction mixture with continuous stirring for about 5 to 15 minutes. The reaction mass that was obtained was then filtered and washed with 10 milliliters of ethyl methyl ketone. The filtrate was taken into a round-bottom flask, which was then subjected to heating to a temperature of about 28° C. with simultaneous stirring. 2.12 grams of maleic acid were dissolved in 12.5 milliliters of ethyl methyl ketone and the resulting solution was added to the above reaction mass at a temperature of about 28° C. over a period of about 30 to 45 minutes with continuous stirring, then the solids were filtered and washed with 10 milliliters of ethyl methyl ketone. The solid mass then obtained was suck dried at temperature of about 28° C., transferred into a petri dish and placed in oven under vacuum for the purpose of drying at a temperature of about 28° C. for about 13 hours. The solid material was further subjected to drying for about 3 hours at a temperature of about 63° C. to afford the compound of interest, tegaserod maleate Form I having a purity of 99.56%, with a yield of 89.5%.

### EXAMPLE IlI

150 liters of methyl ethyl ketone and 5 Kg of tegaserod were placed into a reaction vessel followed by stirring for about 20 to 25 minutes at a temperature of about 25 to 35° C., and the solution was re-circulated through a sparkler filter containing carbon pads for 1 hour. The solution was then transferred into another reaction vessel through a sparkler filter and micron filters, followed by washing the complete filtration system with 30 liters of methyl ethyl ketone. The solution was stirred for about 15 to 20 minutes at temperatures about 25 to 35° C. 2.12 Kg of maleic acid were dissolved in 30 liters of methyl ethyl ketone followed by filtration into another reaction vessel through a filtration system. The maleic acid solution thus obtained was added to the above tegaserod solution that was placed in another reaction vessel over about 10 to 15 minutes at temperatures about 25 to 35° C., followed by stirring for about 30 to 45 minutes at temperatures about 25 to 35° C. The reaction mass was then filtered and washed with 10 liters of methyl ethyl ketone followed by suction drying for about 3 hours. The solid mass was then subjected to drying in a rotary cone vacuum drier at temperatures of 25 to 35° C. under vacuum for about 5 hours. The dried compound was then subjected to size reduction by using a multi miller, then the compound was dried in a rotary cone vacuum drier at temperatures of 25 to 35° C. under vacuum for about 1 hour. Raising the drying temperatures to 60 to 70° C. further dried the compound by means of hot water circulation under vacuum for about 4 hours to get the moisture content at about 0.5%. The compound was then subjected to size reduction by using multi mill and micronized through an air-jet mill at an air pressure of 3.5-4 kg/cm² and finally sifted through a 40-mesh screen to afford the compound of interest, tegaserod maleate Form I, with a yield of 70.2%.

1. A process for preparing crystalline Form I of tegaserod maleate, comprising:
i. dissolving tegaserod in a solvent;
ii. adding a maleic acid solution; and
iii. isolating crystalline Form I of tegaserod maleate.

2. The process of claim 1, further comprising the step of adding carbon to the solution of step (i), absorbing impurities onto the carbon, and removing the carbon.

3. The process of claim 1, wherein the solvent comprises a ketone having the formula R₁C(O)R₂ where R₁ is a straight or branched alkyl group having 1 to 4 carbon atoms, and R₂ is a straight or branched alkyl group having 2 to 4 carbon atoms.

4. The process of claim 1, wherein the solvent comprises methyl isobutyl ketone, methyl isopropyl ketone, ethyl methyl ketone or a mixture of any two or more thereof.

5. The process of claim 2, wherein the solvent comprises ethyl methyl ketone.

6. The process of claim 1, wherein tegaserod. is dissolved at ambient temperatures.

7. The process of claim 1, wherein maleic acid solution is prepared by dissolving maleic acid in a solvent comprising a ketone having the formula R₁C(O)R₂ where R₁ is a straight or branched alkyl group having 1 to 4 carbon atoms, and R₂ is a straight or branched alkyl group having 2 to 4 carbon atoms.

8. The process of claim 7, wherein a solvent for maleic acid comprises methyl isobutyl ketone, methyl isopropyl ketone, ethyl methyl ketone or mixtures thereof.

9. The process of claim 7, wherein a solvent for maleic acid comprises ethyl methyl ketone.

10. The process of claim 7, wherein maleic acid is dissolved at temperatures below about 50° C.

11. The process of claim 7, wherein maleic acid is dissolved at about 25 to about 35° C.

12. The process of claim 1, wherein addition of maleic acid solution is performed at temperatures below about 50° C.

13. The process of claim 1, wherein addition of maleic acid solution is performed at temperatures about 20 to about 45° C.

14. The process of claim 1, wherein addition of maleic acid solution is performed at temperatures about 25 to about 35° C.

15. The process of claim 1, wherein a molar ratio of tegaserod to maleic acid is about 1:1 to about 1:1.5.

16. The process of claim 1, wherein a molar ratio of tegaserod to maleic acid is about 1:1.1.
